(19) United States
(12) Patent Application Publication
(10) Pub. No.: US 2005/0267233 A1
(43) Pub. Date: Dec. 1, 2005

(54) Anti-microbial handle system

(76) Inventor: Ashok V. Joshi, Salt Lake City, UT (US)

Correspondence Address: FACTOR & LAKE, LTD, 1327 W. WASHINGTON BLVD., SUITE 5G/H, CHICAGO, IL, 60607, US

(21) Appl. No.: 10/852,855

(22) Filed: May 25, 2004

(51) Int. Cl. A61K 33/38

(52) U.S. Cl. 523/122; 424/618

(57) ABSTRACT

An antimicrobial handle system comprising a handle and an antimicrobial component in communication with the handle to impart antimicrobial properties thereto. According to particular aspects of the invention, the antimicrobial component may be in the form of an antimicrobial material integrally formed with the handle; a handle portion made from an antimicrobial material and removably connected to the handle; an antimicrobial coating disposed on the handle; a film disposed on the handle; an ion generator arrangement in communication with the handle; a sterilizing gas generator in communication with the handle; a sterilizing corona generator in communication with the handle; an antimicrobial lightsource in communication with the handle; a fluid dispenser to dispense an antimicrobial fluid onto the handle; or a combination thereof. The antimicrobial component may include an active oxygen releasing material; an active ozone, UV light, Halide gas, silver ion, or Halide ion releasing material; an antimicrobial fluid; or a photoactive material that exhibits antimicrobial properties when exposed to a light source. Other antimicrobial components are also contemplated in accordance with the principles of the present invention.

### BACKGROUND OF THE INVENTION

This invention relates to antimicrobial objects, and more particularly to handle systems incorporating antimicrobial mechanisms to facilitate the killing of microorganisms on a handle.

The threat of bacterial contamination and the spread of bacteria in various environments has become an increasingly growing concern in general and especially in environments where sterilization is of particular importance. Bacteria can be spread by a wide range of varying mechanisms. For example, some forms of bacteria can become airborne and spread within a particular environment via heating, ventilating and air-conditioning systems in communication with that environment. Other forms are spread by repeated human contact with various objects within an environment, particularly objects that require human contact to access another environment or space. Objects such as handles, keypads, locking mechanisms, or the like that come into contact with humans during their use create a vehicle with which bacteria can be easily spread from one human to another and allow bacterial infection of humans or further transfer of bacteria to other environments where infection or contamination can take place. This is of particular concern in environments where sterilization is of particular importance, such as in hospitals or other health care facilities, biotechnology and pharmaceutical “clean rooms,” food processing/manufacturing and handling environments, water purification environments, etc. In these environments, handles for doors or other access interfaces are particularly important because they typically are one of the most frequently contacted objects, as well as one of the first objects to be contacted during access to these environments. They are also frequently used within these environments. An antimicrobial handle would therefore be particularly useful in facilitating access to, and use within, such environments while preventing microbial migration and contamination into, and within, these environments.

### SUMMARY OF THE INVENTION

The present invention generally provides an antimicrobial handle system comprising a handle and an antimicrobial component in communication with the handle to impart antimicrobial properties thereto. According to particular aspects of the invention, the antimicrobial component may be in the form of an antimicrobial material integrally formed with the handle; an antimicrobial material disposed on or adjacent to the handle; a handle portion, such as a handle cover, made from an antimicrobial material and removably connected to the handle; an antimicrobial coating disposed on the handle; an antimicrobial film disposed on the handle; an ion generator arrangement in communication with the handle; a sterilizing gas generator in communication with the handle; a sterilizing corona generator in communication with the handle; an antimicrobial lightsource in communication with the handle; or a fluid dispenser to dispense an antimicrobial fluid onto the handle. The antimicrobial component may include an active oxygen releasing material; an active ozone, UV light, Halide gas, silver ion, or Halide ion releasing material; an antimicrobial fluid; or a photoactive material that exhibits antimicrobial properties when exposed to a light source. Other antimicrobial components and compositions are also contemplated in accordance with the principles of the present invention.

According to yet another aspect of the invention, an antimicrobial handle system comprises a handle, an antimicrobial component in communication with the handle, and a sensor in communication with the antimicrobial component. Upon activation of the sensor, the antimicrobial component imparts antimicrobial properties to the handle.

According to yet another aspect of the invention, the system further comprises a controller in communication with the sensor and the antimicrobial component to control the antimicrobial component.

According to particular aspects of the invention, the antimicrobial component may comprise an antimicrobial lightsource and/or a photoactive material that exhibits antimicrobial properties when exposed to the light from the lightsource; an ion generator arrangement in communication with the sensor; or a fluid dispenser containing antimicrobial fluid and in communication with the sensor, wherein the fluid dispenser dispenses the antimicrobial fluid onto the handle upon activation of the sensor.

According to yet another aspect of the invention, a method of killing microorganisms on a handle is contemplated. The method comprises the steps of providing an antimicrobial component in communication with the handle, sensing an event, and imparting antimicrobial properties to the handle via the antimicrobial component upon sensing of the event.

According to yet another aspect of the invention, a method of killing microorganisms on a handle comprises the steps of providing a sterilizing gas generator in communication with the handle, sensing an event, and imparting sterilizing gas to the handle from the gas generator upon sensing of the event.

According to yet another aspect of the invention, a method of killing microorganisms on a handle comprises the steps of providing a sterilizing corona generator in communication with the handle, sensing an event, and imparting a sterilizing corona effect to the handle from the corona generator upon sensing of the event.

These and other aspects of the present invention will be apparent upon review of the written specification, drawings and claims herein.

### BRIEF DESCRIPTION OF THE DRAWINGS

FIG. 1A is a schematic diagram generically depicting a handle and an antimicrobial component in communication with the handle in accordance with the principles of the present invention.

FIG. 1B is a side elevational view of a first embodiment of a handle in accordance with the principles of the present invention.

FIG. 2 is a cross-sectional view of the handle of FIG. 1 taken along section line 2-2 in FIG. 1B.

FIG. 3 is a cross-sectional view of another embodiment of a handle in accordance with the principles of the present invention.

FIG. 4 is a cross-sectional view of yet another embodiment of a handle in accordance with the principles of the present invention.

FIG. 5 is a schematic diagram generically depicting a handle and an antimicrobial component including a light source in communication with an antimicrobial portion of the handle in accordance with the principles of the present invention.

FIG. 6 is a schematic diagram generically depicting a handle and an antimicrobial component including an antimicrobial fluid dispenser in communication with the handle in accordance with the principles of the present invention.

FIG. 7 is a schematic diagram generically depicting a handle and an antimicrobial component including an ionic conductor arrangement in communication with the handle in accordance with the principles of the present invention.

FIG. 8 is a schematic diagram generically depicting a handle and an antimicrobial component including a sterilizing gas generator in communication with the handle in accordance with the principles of the present invention.

FIG. 9 is a schematic diagram generically depicting a handle and an antimicrobial component including a sterilizing corona generator in communication with the handle in accordance with the principles of the present invention.

FIG. 10 is a schematic diagram generically depicting a handle and an antimicrobial component in communication with a sensor in accordance with the principles of the present invention.

FIG. 11 is a schematic diagram generically depicting a handle and an antimicrobial component in communication with a controller and a sensor in accordance with the principles of the present invention.

FIG. 12A is a cross-sectional view of a handle and an antimicrobial component in the form of a handle cover in communication with the handle in accordance with the principles of the present invention.

FIG. 12B is a cross-sectional view of a handle, a handle cover and antimicrobial component in communication with the handle cover in accordance with the principles of the present invention.

### DETAILED DESCRIPTION OF THE PREFERRED EMBODIMENTS

While this invention is capable of embodiment in many different forms, there is shown in the drawings, and will herein be described in detail, one or more specific embodiments with the understanding that the present disclosure is to be considered an exemplification of the principles of the invention and is not intended to limit the invention to these specific embodiments.

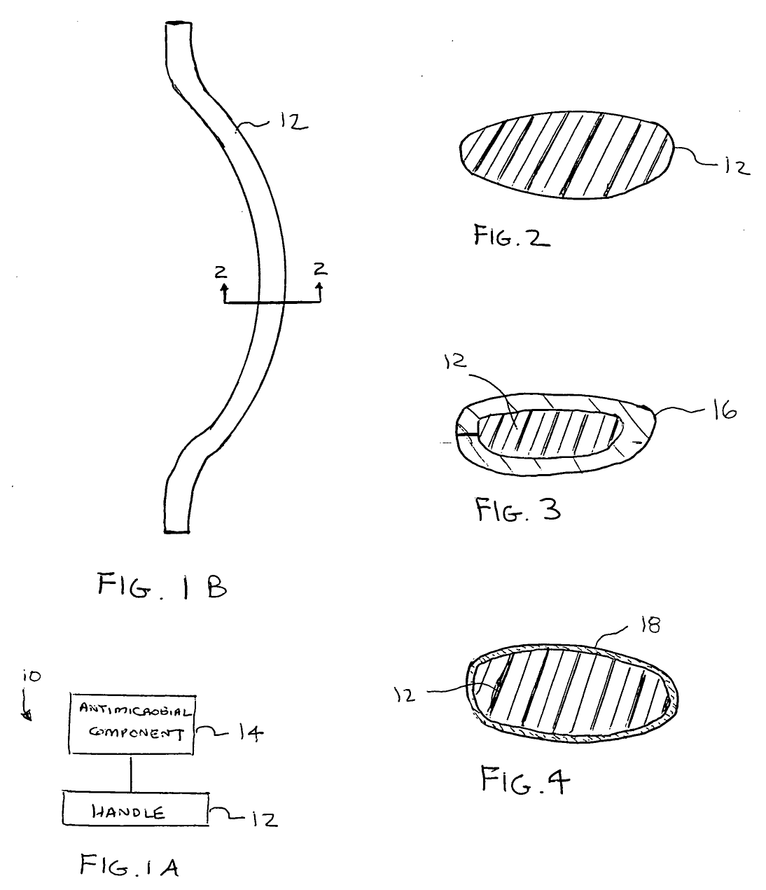

Referring to FIG. 1A, the present invention generally provides an antimicrobial handle system 10 comprising a handle 12 and an antimicrobial component 14 in communication with the handle 12 to impart antimicrobial properties thereto. Referring to FIG. 1B, a handle 12 is generally depicted for purposes of illustration. Although the handle 12 is shown with a given form and shape, it should be understood that the handle 12 may take on any number of form factors in accordance with the principles of the present invention. FIGS. 2-4 generally illustrate some of the forms the antimicrobial component 14 may take. FIG. 2 is a cross-section of the handle 12 taken along section lines 2-2 in FIG. 1B, which illustrates an embodiment wherein the handle 12 includes an antimicrobial component in the form of an antimicrobial material integrally formed with the handle 12. The material forming the handle 12 may be an antimicrobial material itself, or it may be a structural material impregnated or blended with an antimicrobial material. The antimicrobial material may also be chemically bound to the structural material, such as, for example, a polymer matrix having an antimicrobial material bound or complexed thereto.

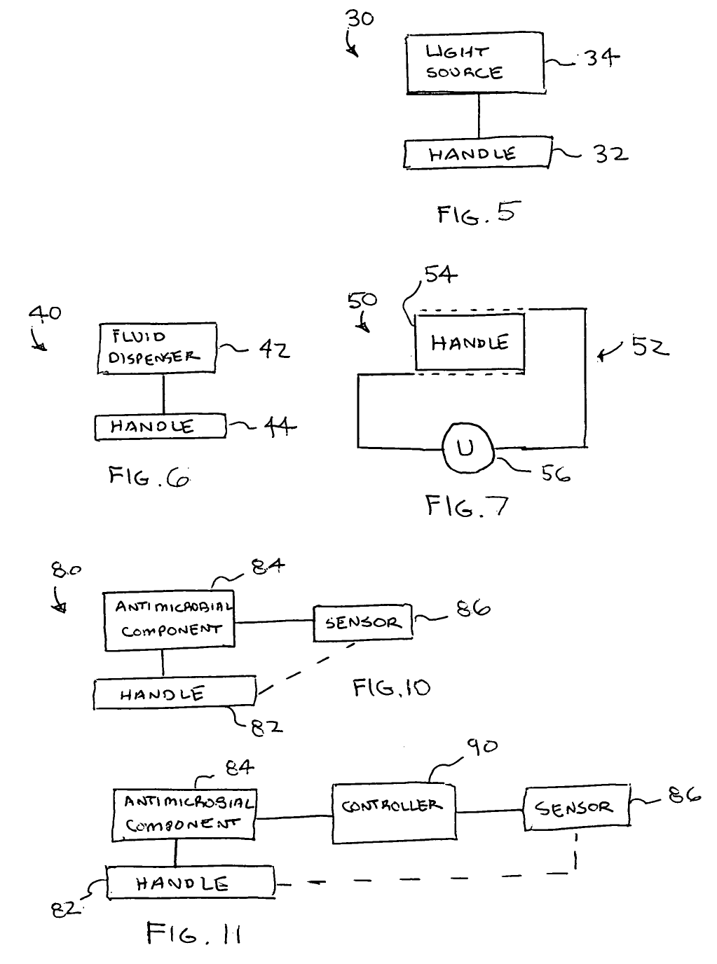

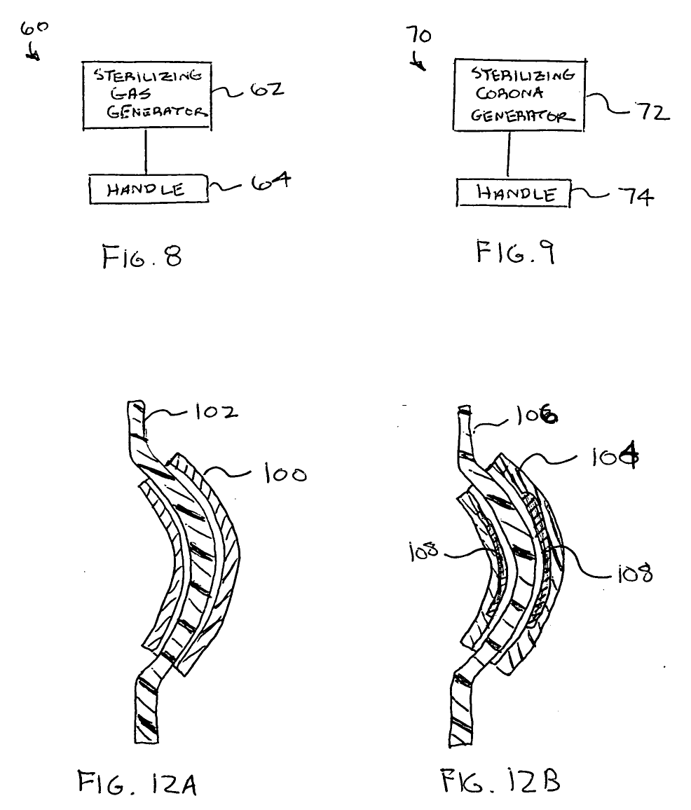

FIGS. 3 and 4 are cross-sectional views similar to FIG. 2 and illustrate other embodiments wherein an antimicrobial material is disposed on or adjacent to the handle 12. For ease of illustration, the handle 12 is referenced by the same reference number in FIGS. 2-4 with the understanding that the handle 12 differs in each figure by what is shown in the cross-sections of each figure. Referring now to FIG. 3, a handle portion 16 made from an antimicrobial material is removably connected to the handle 12. The handle portion 16 may be connected to the handle 12 by a number of means, such as, for example, interference fit, snap fit, mechanically fastened, or overlaid onto the handle 12. The handle portion 16 may also be in the form of a carrier material to accommodate the antimicrobial material, such as, for example, a foam or cloth material impregnated with or containing an antimicrobial agent. In an embodiment, the antimicrobial agent may be in the form of a fluid—such as a peroxide for example—carried by a foam or other cellular material, wherein the fluid is released by pressure applied to the handle 12 by a user.

Referring to FIG. 4, an antimicrobial layer 18 is disposed on the handle 12 in the form of a coating, film or fluid disposed on the handle 12. The antimicrobial layer 18 comprises a material that exhibits or imparts antimicrobial properties. Coatings may applied by numerous techniques, including chemical vapor deposition, physical vapor deposition, dip coating, electrochemical deposition, sputtering, mechanical methods of coating, or the like.

As described, the antimicrobial component may include an antimicrobial material. It is contemplated that numerous materials that exhibit or impart antimicrobial properties can be utilized. In a particular embodiment, the antimicrobial material comprises an active oxygen releasing material. The antimicrobial component may comprise an active oxygen releasing material, active ozone, UV light, Halide gas, silver ion, or Halide ion releasing material, an antimicrobial fluid, or a photoactive material that exhibits antimicrobial properties when exposed to a light source. Materials may include a nonmetal halide; a nonmetal oxide, such as I₂O₅; a ceramic composite and a halide; a water insoluble peroxide; or a water insoluble superoxide, such as CuO, AgO, MgO₂, sulfonated silver polymers, and combinations thereof. In a particular embodiment, the material is of the form ABO_{4+δ}, wherein A is selected from the group consisting essentially of the Larithamide series and B is selected from the group consisting essentially of Cu, Ag, and Au. Other embodiments are contemplated wherein the antimicrobial material comprises an active ozone releasing material, a UV light releasing material, a Halide gas releasing material, a silver ion releasing material, a Halide ion releasing material, or any other material, compound or combination thereof capable of releasing an antimicrobial agent or exhibiting antimicrobial properties.

Particular embodiments of antimicrobial handle systems in accordance with the principles of the present invention will now be described.

Referring to FIG. 5, an antimicrobial handle system 30 may be provided wherein the antimicrobial component includes a handle 32 having a component or portion comprising a photosensitive material that imparts antimicrobial properties to the handle 32 when it is exposed to a light source 34. Examples of such materials include silver halides, copper halides, silver and aluminum. The light source 34 may be separate from, or integrated with the handle 32. In an embodiment, the light source 34 is in the form of an LED that is integrated into the handle 32 such that, when the LED is illuminated, antimicrobial properties are imparted to the handle 32 by the photosensitive material being exposed to the light from the LED. Other light sources having various wavelength outputs are contemplated as well, depending on the photosensitive material implemented. Other embodiments may incorporate an antimicrobial light source without the use of a photosensitive material, such as, for example, a UV light source that provides UV light, which is known to exhibit antimicrobial effects. Preferably, the UV light source radiates light having a wavelength in the range of about 150 nm to about 400 nm. In another embodiment, the light source comprises a halogen generating source.

Referring to FIG. 6, an antimicrobial handle system 40 may be provided wherein the antimicrobial component includes a fluid dispenser 42 for dispensing an antimicrobial fluid to a handle 44 in communication therewith. The dispenser 42 may be implemented in a number of forms and may be integrated with, or separate from, the handle 44. In an embodiment, the dispenser 42 may be integrated with the handle 44 and in communication with a pressure source such that when pressure is supplied, fluid is dispensed onto the handle 44 in a predetermined amount. In an embodiment, pressure may be supplied by squeezing a portion of the handle 44 and/or dispenser 42 by hand. The antimicrobial fluid may comprise an antimicrobial gas or an antimicrobial liquid. In another embodiment, the dispenser dispenses an antimicrobial agent in solid form, such as an antimicrobial powder.

Referring to FIG. 7, an antimicrobial handle system 50 may be provided wherein the antimicrobial component includes an ion generator in the form of an ionic conducting arrangement 52 in communication with a handle 54. In this arrangement, antimicrobial properties are imparted to the handle 54 via generation of ions through ionic conduction, wherein ions concentrate on a surface of the handle 54. Ionic conducting materials suitable for this arrangement may include Ag₄PbI₅, AgI—Al₂O₃ composite, PbI₂ chloride conductive compounds, iodine conductive compounds, fluoride conductive compounds, or any other material, compound or combination of materials or compounds capable of ionic conduction. Ionic conduction is facilitated by a source potential 56 that is preferably integrated into the handle 54. The source potential 56 may also be a component separate from the handle 54 and in communication therewith. Other forms of ion generators known in the art can be implemented as well in accordance with the principles of the present invention to facilitate generation of ions to impart antimicrobial properties to the handle 54.

Referring to FIG. 8, an antimicrobial handle system 60 may be provided wherein the antimicrobial component includes a sterilizing gas generator 62 in communication with a handle 64. In this arrangement, antimicrobial properties are imparted to the handle 64 by imparting a sterilizing gas to the handle 64 from the gas generator 62. Any type of sterilizing gas known in the art to have antimicrobial properties can be implemented in accordance with the principles of the present invention to facilitate imparting of antimicrobial properties to the handle 64. The gas generator 62 can be positioned adjacent to the handle or incorporated into the handle so that it can be in communication with the handle to effectively impart antimicrobial properties to the handle 64.

Referring to FIG. 9, an antimicrobial handle system 70 may be provided wherein the antimicrobial component includes a sterilizing corona generator 72, such as a corona cell, in communication with a handle 74. In this arrangement, antimicrobial properties are imparted to the handle 74 by imparting a sterilizing corona effect to the handle 74 from the corona generator 72. Corona generators, such as corona cells, known in the art to have antimicrobial effects can be implemented in accordance with the principles of the present invention to facilitate imparting of antimicrobial properties to the handle 74. The corona generator 72 can be positioned adjacent to the handle or incorporated into the handle so that it can be in communication with the handle to effectively impart antimicrobial properties to the handle 74.

According to another aspect of the invention, and generally referring to FIG. 10, an antimicrobial handle system 80 is provided that includes a handle 82 and an antimicrobial component 84 in communication with the handle 82 that imparts antimicrobial properties thereto upon occurrence of an event. In a preferred embodiment, as shown in FIG. 10, the event may be sensed by a sensor 86 in communication with the antimicrobial component 84 of the system. Referring to FIG. 10, the antimicrobial component 84 is in communication with the handle 82 and the sensor 86 is in communication with the antimicrobial component 84 such that upon activation of the sensor 86, the antimicrobial component 84 imparts antimicrobial properties to the handle 82. For example, in an embodiment utilizing an antimicrobial material in the form of a photosensitive material incorporated into a handle, such as that illustrated in FIG. 5, the sensor 86 may be a motion or proximity sensor that senses an approaching person and activates the light source to expose the photosensitive material to light. Thus, the light source will activate the antimicrobial component of the handle prior to the handle being touched by the approaching person. The sensor 86 may also be a touch-type sensor that is activated upon touch. The sensor arrangement can be implemented in any of the aforementioned embodiments to activate the antimicrobial component upon activation of the sensor.

As an extension of the embodiment shown in FIG. 10, FIG. 11 depicts a similar system but further including a controller 90 in communication with the antimicrobial component 84 and the sensor 86 for controlling the antimicrobial component 84, e.g., a light source (FIG. 5), a fluid dispenser (FIG. 6), an ion generating arrangement (FIG. 7), a sterilizing gas generating arrangement (FIG. 8), or a sterilizing corona generating arrangement (FIG. 9). The dotted line between the sensor 86 and the handle in both FIGS. 10 and 11 indicate that the sensor 86 may be integrated into the handle or in communication therewith.

As already noted with respect to the embodiment generically depicted in FIG. 3, the handle portion 16 is made from an antimicrobial material and is removably connected to the handle 12. As a further extension of this embodiment, FIG. 12A more specifically shows an embodiment incorporating a handle cover 100 in communication with a handle 102. The handle cover 100 preferably comprises a metal mesh or metal screen material formed to cover the handle 102. The metal mesh material can exhibit antimicrobial properties itself, i.e., have an antimicrobial agent integrated therewith, and thus act as an antimicrobial component of the handle system. Alternatively, the handle cover 100 may include an antimicrobial agent or mechanism in communication therewith. The handle cover 100 shown in FIG. 12A includes an antimicrobial component integrated into the handle cover 100. The handle cover 100 may incorporate a metal mesh of Ni, Fe, Zn, Sn, Cr or Mg and coated with an antimicrobial inorganic material, such as oxides of silver or halogen; halides of metals; photoactive antimicrobial materials such as silver halides, TiO₂, SnO₂, and combinations thereof; or peroxides such as MgO₂, silver peroxides and iodine pentoxide.

FIG. 12B depicts an embodiment wherein a handle cover 104 of a handle 106 incorporates an antimicrobial component in communication therewith. In this embodiment, the cover 104 may include an antimicrobial material generator 108, such as a corona generator, a negative ion generator such as oxygen ion or hydroxyl ion, a positive ion generator such as metal ions, an ozone generator, an electron generator, or an anti-microbial fluid delivery system.

While specific embodiments have been illustrated and described, numerous modifications may come to mind without significantly departing from the spirit of the invention, and the scope of protection is only limited by the scope of the accompanying claims.

1. An antimicrobial handle system comprising:
a handle; and
an antimicrobial component in communication with the handle to impart antimicrobial properties thereto.

2. The handle system of claim 1, wherein the antimicrobial component comprises an antimicrobial material integrally formed with the handle.

3. The handle system of claim 1, wherein the antimicrobial component comprises a handle portion made from an antimicrobial material and removably connected to the handle.

4. The handle system of claim 3, wherein the handle portion comprises a handle cover.

5. The handle system of claim 4, wherein the handle cover comprises a metal screen formed to cover the handle.

6. The handle system of claim 4, wherein the handle cover is made of a material selected from the group consisting of Fe, Ni, Zn, Ti, and combinations thereof.

7. The handle system of claim 4, wherein the handle cover is made of a material selected from the group consisting of a halide and an oxide of silver.

8. The handle system of claim 1, wherein the antimicrobial component comprises an antimicrobial coating disposed on the handle.

9. The handle system of claim 1, wherein the antimicrobial component comprises a film disposed on the handle.

10. The handle system of claim 1, wherein the antimicrobial component comprises an active oxygen releasing material.

11. The handle system of claim 1, wherein the antimicrobial component comprises an antimicrobial fluid.

12. The handle system of claim 1, wherein the antimicrobial component comprises a nonmetal halide.

13. The handle system of claim 1, wherein the antimicrobial component comprises a nonmetal oxide.

14. The handle system of claim 13, wherein the nonmetal oxide comprises I₂O₅.

15. The handle system of claim 1, wherein the antimicrobial component comprises a ceramic composite and a halide.

16. The handle system of claim 1, wherein the antimicrobial component comprises a material of the form ABO_{4+δ}, wherein A is selected from the group consisting essentially of the Larithamide series and B is selected from the group consisting essentially of Cu, Ag, and Au.

17. The handle system of claim 1, wherein the antimicrobial component consists essentially of a water insoluble peroxide.

18. The handle system of claim 1, wherein the antimicrobial component consists essentially of a water insoluble superoxide.

19. The handle system of claim 1, wherein the antimicrobial component consists essentially of a superoxide selected from the group consisting essentially of CuO, AgO, MgO₂, sulfonated silver polymers, and combinations thereof.

20. The handle system of claim 1, wherein the antimicrobial component comprises a photoactive material that exhibits antimicrobial properties when exposed to light.

21. The handle system of claim 20, further comprising a light source in communication with the handle.

22. The handle system of claim 21, further comprising a sensor in communication with the light source to activate the light source upon sensing of an event.

23. The handle system of claim 21, wherein the sensor comprises a motion sensor.

24. The handle system of claim 21, further comprising a controller in communication with the light source to control the light source.

25. The handle system of claim 21, wherein the light source comprises a halogen generating source.

26. The handle system of claim 21, wherein the light source comprises an ultraviolet light generating source.

27. The handle system of claim 26, wherein the ultraviolet light generating source generates light having a wavelength between about 150 nm to about 400 nm.

28. The handle system of claim 1, wherein the antimicrobial component comprises an ion generating arrangement in communication with the handle.

29. The handle system of claim 28, further comprising a sensor in communication with the ion generating arrangement to activate the ion generating arrangement upon sensing of an event.

30. The handle system of claim 28, wherein the ion generating arrangement comprises an ionic conductor arrangement.

31. The handle system of claim 1, wherein the antimicrobial component comprises an antimicrobial fluid, and the system further comprises a fluid dispenser to dispense the antimicrobial fluid onto the handle.

32. The handle system of claim 31, wherein the antimicrobial fluid comprises a fluid selected from the group consisting essentially of an antimicrobial gas, an antimicrobial liquid, an antimicrobial solid in powder form, and combinations thereof.

33. The handle system of claim 32, further comprising a sensor in communication with the fluid dispenser to activate dispensing of the antimicrobial fluid upon sensing of an event.

34. The handle system of claim 33, further comprising a controller in communication with the fluid dispenser to control dispensing of the antimicrobial fluid.

35. The handle system of claim 1, wherein the antimicrobial component comprises a sterilizing gas generating arrangement in communication with the handle.

36. The handle system of claim 35, further comprising a sensor in communication with the sterilizing gas generating arrangement to activate the sterilizing gas generating arrangement upon sensing of an event.

37. The handle system of claim 35, wherein the sterilizing gas generating arrangement comprises a sterilizing gas generating cell.

38. The handle system of claim 1, wherein the antimicrobial component comprises a corona generating arrangement in communication with the handle.

39. The handle system of claim 38, further comprising a sensor in communication with the corona generating arrangement to activate the corona generating arrangement upon sensing of an event.

40. The handle system of claim 38, wherein the corona generating arrangement comprises a corona generating cell.

41. An antimicrobial handle system comprising:
a handle; and
an antimicrobial component in communication with the handle that imparts antimicrobial properties thereto upon occurrence of an event.

42. The handle system of claim 41, wherein the event is sensed by a sensor in communication with the antimicrobial component.

43. The handle system of claim 42, wherein the sensor is a pressure transducer in communication with the handle that senses pressure applied to the handle.

44. The handle system of claim 42, wherein the sensor is a motion sensor.

45. An antimicrobial handle system comprising:
a handle;
an antimicrobial component in communication with the handle; and
a sensor in communication with the antimicrobial component such that upon activation of the sensor, the antimicrobial component imparts antimicrobial properties to the handle.

46. The handle system of claim 45, wherein the antimicrobial component is selected from the group consisting essentially of light, a gas, a liquid, a solid, and combinations thereof.

47. The handle system of claim 45, wherein the antimicrobial component is an oxidizing liquid selected from the group consisting essentially of a hydrogen peroxide solution, an alcohol solution, and combinations thereof.

48. The handle system of claim 45, wherein the antimicrobial component is a gas selected from the group consisting essentially of active oxygen, ozone, halogen, and combinations thereof.

49. The handle system of claim 45, wherein the antimicrobial component is a solid selected from the group consisting essentially of a silver oxide, a silver halide, a non-metal halide, a non-metal oxide, a water insoluble super oxide, and combinations thereof.

50. The handle system of claim 45, further comprising a controller in communication with the sensor and the antimicrobial component to control the antimicrobial component.

51. The handle system of claim 45, wherein the antimicrobial component comprises a lightsource and a photoactive material that exhibits antimicrobial properties when exposed to the light from the lightsource.

52. The handle system of claim 45, wherein the antimicrobial component comprises an ionic conductor arrangement in communication with the sensor.

53. The handle system of claim 45, further comprising a fluid dispenser containing antimicrobial fluid and in communication with the sensor, wherein the fluid dispenser dispenses the antimicrobial fluid onto the handle upon activation of the sensor.

54. A method of killing microorganisms on a handle, the method comprising the steps of:
providing an antimicrobial component in communication with the handle;
sensing an event;
imparting antimicrobial properties to the handle via the antimicrobial component upon sensing of the event.

55. An antimicrobial handle system comprising:
a handle;
an antimicrobial component in communication with the handle;
a sensor in communication with the antimicrobial component; and
a controller in communication with the sensor and the antimicrobial component to control the antimicrobial component to impart antimicrobial properties to the handle upon activation of the sensor.

56. The handle system of claim 55, wherein the antimicrobial component comprises a light source and a photoactive material that exhibits antimicrobial properties when exposed to the light from the light source.

57. The handle system of claim 56, wherein the light source is activated by the sensor to expose the photoactive material to light.

58. The handle system of claim 56, wherein the light source and the photoactive material are integrated into the handle.

59. The handle system of claim 55, wherein the antimicrobial component is integrated into the handle.

60. The handle system of claim 55, wherein the antimicrobial component comprises an ionic conductor arrangement in communication with the controller to control the ionic conductor arrangement to impart antimicrobial properties to the handle upon activation of the sensor.
